(19) United States
(12) Patent Application Publication
(10) Pub. No.: US 2017/0354799 A1
(43) Pub. Date: Dec. 14, 2017

(54) CANNULA NEEDLE

(71) Applicants: Deepak Gupta, Detroit, MI (US); Sarwan Kumar, Troy, MI (US); Arvind Sri Raja Kalidindi, Troy, MI (US)

(72) Inventors: Deepak Gupta, Detroit, MI (US); Sarwan Kumar, Troy, MI (US); Arvind Sri Raja Kalidindi, Troy, MI (US)

(21) Appl. No.: 15/195,351

(22) Filed: Jun. 28, 2016

Related U.S. Application Data
(60) Provisional application No. 62/348,425, filed on Jun. 10, 2016.

(51) Int. Cl. A61M 25/06

(52) U.S. Cl. CPC A61M 25/0606; A61M 25/065; A61M 25/0693; A61M 2210/12

(57) ABSTRACT

According to a teaching of the present invention, having a flexible catheter attached to a housing. A needle having an exterior surface having a first roughness, the needle being within the flexible catheter and the housing. The needle has and interface surface angled with respect to a needle centerline. A portion of the interface surface having a second roughness which is more than the first roughness.

### CROSS-REFERENCE TO RELATED APPLICATIONS

This application claims the benefit of U.S. Provisional Application No. 62/348,425, filed on Jun. 10, 2016. The entire disclosure of the above application is incorporated herein by reference.

### FIELD

The present invention relates to the needles for intravenous (IV) catheters, and in particular, the invention relates to a needle puncturing surface which facilitates the flow of blood to improve a visible indication of the timing and presence of a proper IV needle insertion.

### BACKGROUND

This section provides background information related to the present disclosure which is not necessarily prior art.

Often IV catheters are used to maintain fluid & electrolytes balance, to restore circulating volume, to administer medications, to transfuse blood & blood products and to correct or maintain the patient's nutritional status. Isotonic fluids, which should be used with caution, use with caution with congestive heart failure, renal dysfunction & circulatory insufficiency include Sodium chloride 0.9% solution and Lactated Ringer's Solution. Also used are hypotonic solutions which include Sodium chloride 0.45% solution. D 5% in 0.45% NS; and Hyper Tonic Fluids that must be administer cautiously & only when the serum osmolality has decreased to dangerously low levels. D 10% in water. D 50% in water, Crystalloid, NR+Colloid; and blood products. Transitionally, these liquids are introduced to the patient using an IV catheter.

Factors to be consider when choosing an IV site including the condition of the vein, the types of IV therapy, and the duration of IV therapy. Additionally, important are the patient's age & size, whether the patient is right or left handed, and the skills of person performing the vein puncture. Often, IV catheters are placed in the veins of the hand such as 1—digital dorsal veins; 2—dorsal metacarpal veins; 3—dorsal venous network; 4—cephalic vein; and 5—basilic vein. Additionally, they can be placed in the veins of the forearm including 1—cephalic vein; 2—median cubital vein; 3—accessory cephalic vein; 4—basilic vein; 5—cephalic vein; and 6—median antebrachial vein.

While it is preferred to use the veins in the upper extremities as opposed to that of the lower extremity sites for IV cannulation. Because the superficial veins of lower extremity connected directly to the deeper veins in the leg there is a high risk of DVT & phlebitis. This site should be restricted to short term administration only and changed as soon as other site can be established elsewhere.

Unfortunately, due to physiology, it is often difficult to properly place the insertion needle into the vein. In this regard, it is unfortunately possible to have the insertion needle to be placed through the receiving vein causing discomfort and bruising. It is therefore a primary objective of the invention to provide an IV catheter device in which the disadvantages of the prior art are overcome. Therefore, the foregoing objectives, other objects, as well as numerous advantages of the present invention, are set forth in the following disclosure.

### SUMMARY OF THE INVENTION

The foregoing objectives may be achieved by a needle device for an intravenous catheter apparatus having a needle it configured to facilitate the flow of blood to improve a visible indication of the timing and presence of a proper IV needle insertion.

According to a teaching of the present invention, having a flexible catheter attached to a housing. A needle having an exterior surface having a first roughness, the needle being within the flexible catheter and the housing. The needle has and interface surface angled with respect to a needle centerline. A portion of the interface surface having a second roughness which is more than the first roughness.

According to a teaching of the present invention, the intravenous catheter apparatus above wherein second roughness has a Ra of 0.2 to 1.6 um.

According to a teaching of the present invention, the intravenous catheter apparatus above wherein the exterior surface defined a first channel.

According to a teaching of the present invention, the intravenous catheter apparatus above wherein the interface surface defines a second channel.

According to a teaching of the present invention, the intravenous catheter apparatus above further has a member defining a flashback chamber.

According to a teaching of the present invention, the intravenous catheter apparatus above further has a catheter base within the wing housing.

According to a teaching of the present invention, the intravenous catheter apparatus above wherein the needle safety device is capable of receiving between 14 through 18 gauge needles.

According to a teaching of the present invention, the intravenous catheter apparatus above wherein the catheter is capable of receiving between 18 through 24 gauge needles.

According to another feature of the present invention, the intravenous catheter apparatus has cylindrical wing housing. The needle according to the present teachings fitting efficiently within the cylindrical wing housing.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The drawings described herein are for illustrative purposes only of selected embodiments and not all possible implementations, and are not intended to limit the scope of the present disclosure.

FIG. 1 is a perspective view of an intravenous catheter apparatus according to the present teachings;

FIG. 2 is a cross sectional view of the apparatus of FIG. 1 taken along line 2-2;

FIGS. 3A-3D are cross sectional views of the apparatus of FIG. 1 taken along line 3-3; and

FIGS. 4A-E represent tips of the needle shown in FIGS. 1-3D according to the present teachings.

Corresponding reference numerals indicate corresponding parts throughout the several views of the drawings.

### DETAILED DESCRIPTION OF THE PREFERRED EMBODIMENT

Example embodiments will now be described more fully with reference to the accompanying drawings.

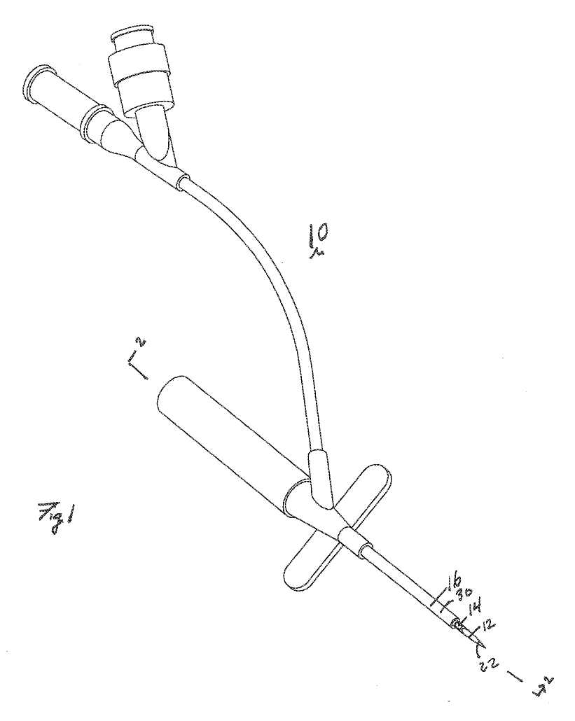

According to FIGS. 1-4D, an IV catheter system 10 according to the present teachings is disclosed. The system 10 consists of a needle 12 inside a plastic catheter 14 which is inserted into the vascular system during a medical procedure. The catheter 14 and needle assembly can be supported by a cannula 16. The needle 12 is used to enter the skin & the vein and provides a stable support structure during the insertion of the catheter 14 within the vein. The needle 12 is removed once the cannulation is accomplished. The plastic catheter 14 which, is soft & flexible, is the only thing left within the vein and has the advantage of being secure & stable in the vein. The catheter 14 is supported by the annularly disposed cannula 16, which is available in a variety of designs & sizes, with wings or without wings.

Typically, the assembly 10 has a steel needle 12, plastic wings & a flexible extension cannula tube 16 that varies in length from 3 to 12 inches. The needle size is ranging from 0.5 to 1 inch, & gauges from 17 to 27. It is suitable for short term IV therapy (less than 24 hrs), obtaining blood sample, with infants, pediatric, & elderly patients. For trauma patient & major surgery 18 g. For blood transfusion, 20 g. For most adult patients a 22 g. needle can be used. For pediatric & elderly patients with fragile veins, a 24 g. can be appropriate.

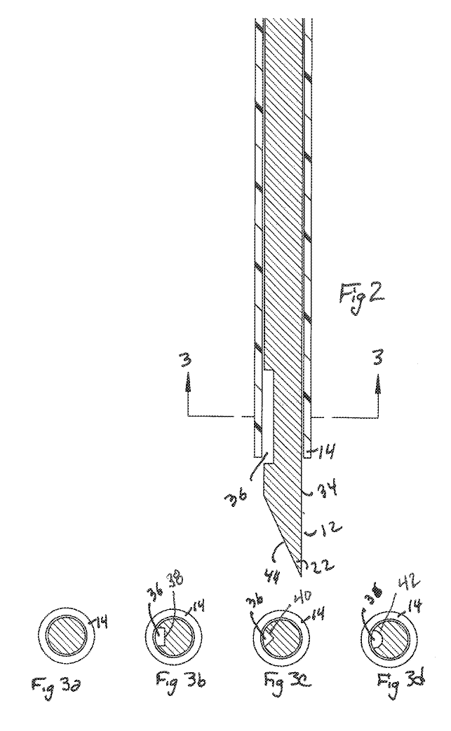

As shown in FIG. 1, an intravenous (IV) catheter apparatus 10 of the present invention is shown. The IV catheter apparatus 10 includes the needle 12, a needle cover, a wing housing, a port cap in unitary assembly with the wing housing. As seen in the cross section depicted in FIG. 2, a needle 12 extends from the cannula 16 through the catheter 14 and ends in a needle tip 22 under the needle cover. As described in detail below, the needle 12 can define a hollow groove portion which facilitates a small amount of blood which is visible with a transparent portion of a flash back chamber 30 once the needle 12 pierces a vein. The needle tip 22 is typically cut at a diagonal. When the needle tip 22 is inserted into a patient the needle may encounter fluid pressure which travels adjacent needle tip 22 and the needle 12 and in a space defined between the needle 12 and the catheter 14 and enter a flash back chamber 30 which give a visual indication of a proper insertion.

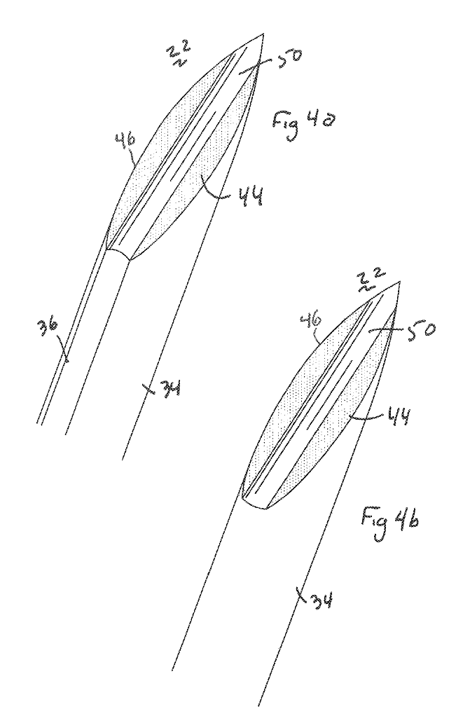

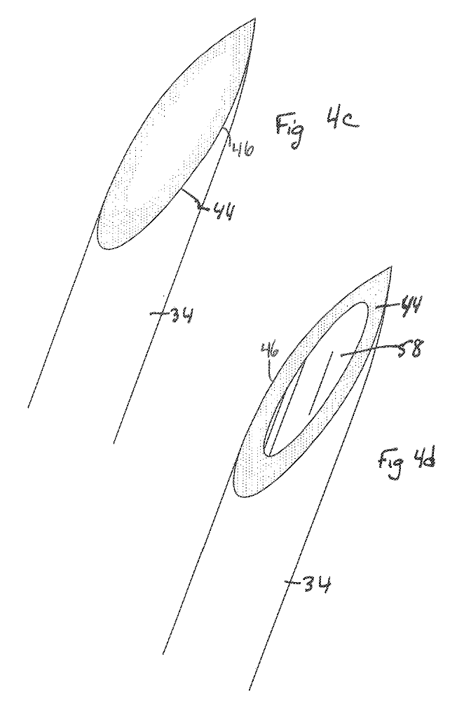

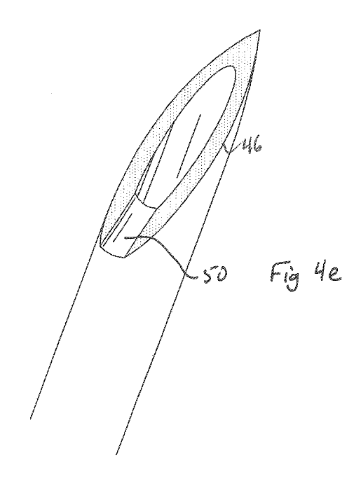

As shown in FIGS. 3a-3d, surrounding the needle is a catheter 14 which is attached to the wing housing or catheter 14 by catheter base 28. The catheter 14 is a flexible tube that will remain in the user to provide fluids, nourishment, or medicines. As shown, the exterior surface 34 of the needle 12 can define a channel 36. The exterior surface 34 has a generally polished finish having a first roughness Ra of 0.1 to 0.6 um. The channel 36 can have a flat lower surface 38, an angled surface 40, or a rounded channel 42. The channel 36 can help facilitate the flow of indicating blood to the flashback chamber.

As shown in FIGS. 4A-4E, the needle tip can be formed by an angled cut that forms a generally planar interface surface 44. The interface surface 44 is generally oval in shape and has an interface edge 46 between the interface surface 44 and the needle surface 34. The interface edge 46 is roughened compared to the exterior surface 34 to provide a serrated edge which helps facilitate the insertion of the needle 12 in the vein. The interface surface 44 can define a second channel 50 which helps to facilitate the flow of blood to the flashback chamber. As shown in FIG. 4A, the second channel 50 can be fluidly coupled to the first channel or to the need exterior surface 34. The interface surface 44 has a generally finish having a second roughness which is more than the first roughness and has an Ra of 0.2 to 1.6 um.

As shown in FIG. 4c, the interface surface can have a central portion have a third finish having a third roughness which is less than the second roughness and has an Ra of for example 0.1 to 0.6 um. FIGS. 4D and 4E represent needles 12 defining a shallow through cavity 58 which facilitates the flow of blood to the flashback chamber via portions illustrated in FIGS. 3A-3D.

In use, a medical professional receives the intravenous (IV) catheter apparatus with the needle tip 22 as seen in FIGS. 1-3D, the needle 10 and catheter 12 are inserted into a patient's vein slowly until blood is seen in the flashback chamber. This prevents the needle tip 22 from passing through the obverse side of the vein. The needle 12 is then withdrawn from the patient by grasping the cannula and pulling upon the needle leaving the cannula within the vein. The needle 10 slides through the cannula 16.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. An intravenous catheter apparatus comprising:
a flexible catheter attached to a housing;
a needle having an exterior surface having a first roughness, the needle being within the flexible catheter and the housing, the needle having and interface surface angled with respect to a needle centerline, a portion of the interface surface having a second roughness which is more than the first roughness.

2. The intravenous catheter apparatus of claim 1 wherein second roughness has an Ra of 0.2 to 1.6 um.

3. The intravenous catheter apparatus of claim 1 wherein the exterior surface defined a first channel.

4. The intravenous catheter apparatus of claim 1 wherein the interface surface defines a second channel.

5. The intravenous catheter apparatus of claim 1 further comprising a member defining a flashback chamber.

6. The intravenous catheter apparatus of claim 1 further comprising a catheter base within the wing housing.

7. An intravenous catheter apparatus comprising:
a flexible catheter;
a needle disposed in the flexible catheter, the needle having an exterior surface having a first roughness Ra of 0.2 to 0.6 um, the needle having and interface surface angled with respect to a needle centerline, a portion of the interface surface having a second roughness which is more than the first roughness.

8. The intravenous catheter apparatus of claim 7 wherein second roughness has an Ra of 0.2 to 1.6 um.

9. The intravenous catheter apparatus of claim 7 wherein the exterior surface defined a first channel.

10. The intravenous catheter apparatus of claim 7 wherein the interface surface defines a second channel.

11. The intravenous catheter apparatus of claim 7 further comprising a member defining a flashback chamber.

12. The intravenous catheter apparatus of claim 7 further comprising a catheter base within the wing housing.
